(19) United States
(12) Patent Application Publication
(10) Pub. No.: US 2010/0076789 A1
(43) Pub. Date: Mar. 25, 2010

(54) Method for remote consultation via mobile communication apparatus and system thereof

(76) Inventor: William Pan, Taipei (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 12/591,514

(22) Filed: Nov. 23, 2009

Related U.S. Application Data
(63) Continuation-in-part of application No. 10/801,709, filed on Mar. 17, 2004.

(51) Int. Cl. G06Q 50/00; G06F 3/048; A61B 5/05

(52) U.S. Cl. 705/3; 715/764; 600/407

(57) ABSTRACT

A system for remote consultation via a remote mobile communication apparatus has a medical equipment device, a medical server and a remote mobile communication apparatus. A method for remote consultation employs the medical equipment device to produce the first medical report and store it into the medical server, transmits the first medical report with the medical server to the remote mobile communication apparatus via a wireless communication network, browses the first medical report, increases, modifies or varies the medical image or medical text of the first medical report to form the second medical report, and uses the mobile communication apparatus to send the second medical report back to the medical server via the wireless communication network. The remote consultation can be reached.

### CROSS REFERENCE TO RELATED APPLICATION

This application is a continuation-in-part of U.S. application Ser. No. 10/801,709, filed on 17 Mar. 2004 and entitled “Method for Remote Consultation via Mobile Communication Apparatus and System Thereof”, now pending.

### FIELD OF THE INVENTION

The present invention is directed to a method for remote consultation via a mobile communication apparatus and a system thereof, and more particularly, to a method and system that is used to transmit medical reports from a hospital to a remote mobile communication apparatus for remote consultation.

### BACKGROUND OF THE INVENTION

With the progress in computer science, computer technology has been applied to the medical equipment to improve medical techniques. For instance, the 3-D image techniques have been applied in the ultrasound instrument to obtain the inner image of the human body and the face or image of a baby. Further, the computer technology is also applied to the tomography scanner or endoscopic camera to digitalize the images obtained for storage or further application. Employing the computer to compose the images can quickly reveal the cause of disease and make the success rate of a medical operation increase considerably.

However, in some medical processes or before an important operation, generally, several experienced doctors will need to consult with each other to decide the medical method or operation method. The traditional way is to invite local, national or international, well-known doctors to consult together at a specific time, but this consumes time very much. Besides, for some urgent medical cases, this way usually impedes optimal medical timing.

Further, the mobile communication apparatus, such as cellular phone, is quite popular at present. With the progress of communication techniques and the increase of the transmission bandwidth, some novel cellular phones can be used to send multimedia messages and can even download Java programs (mobile Java program) to increase their functions. The multimedia messages include, for example, various color pictures, animations and sounds.

Since the Java programs can be executed on different platforms, have compact machine codes suited for network transmission and have built-in security checking functions, there is a big communication company proposing Java 2 micro edition (J2ME) mobile information device profile (MIDP) as a standard mobile phone platform for developing Java programs. The software provider can use this platform to develop various application services for user to download.

Hence, by using the multimedia message service (MMS) and the mobile Java programs, the medical images mentioned above can be sent to the cellular phones of the doctors as multimedia messages or sent to a medical apparatus via the cellular phone for emergent consultation. Further, the doctors can also send back a medical report by the cellular phone. The traditional drawbacks can be removed.

### SUMMARY OF THE INVENTION

An objective of the present invention is to provide a method and system for remote consultation via a mobile communication apparatus to solve the conventional problem that doctors can't remotely perform an emergency medical consultation.

For reaching the objective above, the present invention mainly provides a method for remote consultation via a remote mobile communication apparatus. A medical equipment device is employed to produce the first medical report and store it in a medical server. The medical server transmits the first medical report to the remote mobile communication apparatus via a wireless communication network and displays it on a screen. The first medical report is browsed via the mobile communication apparatus. After consultation, the mobile communication apparatus is employed to increase, modify or vary the medical image or medical text of the first medical report to form the second medical report. The mobile communication apparatus is then employed to send the second medical report back to the medical server via the wireless communication network. The remote consultation can thus be conducted.

Another feature of the present invention is to provide the method for remote consultation via a remote mobile communication apparatus mentioned above, in which the mobile communication apparatus further connects with a remote medical apparatus. After employing the medical server to transmit the first medical report to the remote mobile communication apparatus, the first medical report is sent to the remote medical apparatus via the mobile communication apparatus. The first medical report is browsed via the remote medical apparatus. After consultation, the remote medical apparatus is employed to increase, modify or vary the medical image or medical text of the first medical report to form the second medical report. The second medical report is sent to the mobile communication apparatus, and the mobile communication apparatus sends the second medical report back to the medical server via the wireless communication network. The remote consultation can thus be conducted.

Still another feature of the present invention is to provide a system for remote consultation via a remote mobile communication apparatus, and comprises at least a medical equipment device, a medical server, and a remote mobile communication apparatus. The medical equipment device is used to produce a first medical report, the medical server is used for storing the first medical report and the remote mobile communication apparatus is able to connect with the medical server via a wireless communication network for accessing the first medical report for remote consultation and for sending a second medical report back after the second medical report is produced. The remote consultation can thus be conducted.

Still another feature of the present invention is to provide the system for remote consultation via the remote mobile communication apparatus mentioned above, which further includes a remote medical apparatus able to connect with the mobile communication apparatus for accessing the first medical report, producing the second medical report and sending the second medical report back to the mobile communication apparatus. Thereby, it can resolve the problem that the mobile communication apparatus is not convenient to operate for consultation.

Numerous additional features, benefits and details of the present invention are described in the detailed description, which follows.

### BRIEF DESCRIPTION OF THE DRAWINGS

The foregoing aspects and many of the attendant advantages of this invention will be more readily appreciated as the same becomes better understood by reference to the following detailed description, when taken in conjunction with the accompanying drawings, wherein:

FIG. 1 is a schematic diagram of the first embodiment in accord with the present invention;

FIG. 2 is schematic diagram of an inner structure of the medical server in accordance with the present invention;

FIG. 3 is a schematic diagram of the second embodiment in accord with the present invention;

FIG. 4 is a flowchart of the first embodiment of the method for remote consultation in accordance with the present invention;

FIG. 5 is a detailed flowchart of the first embodiment that explains the transmitting of the medical report in accordance with the present invention; and

FIG. 6 is a detailed flowchart of the second embodiment that explains the transmitting of the medical report in accordance with the present invention.

### DETAILED DESCRIPTION

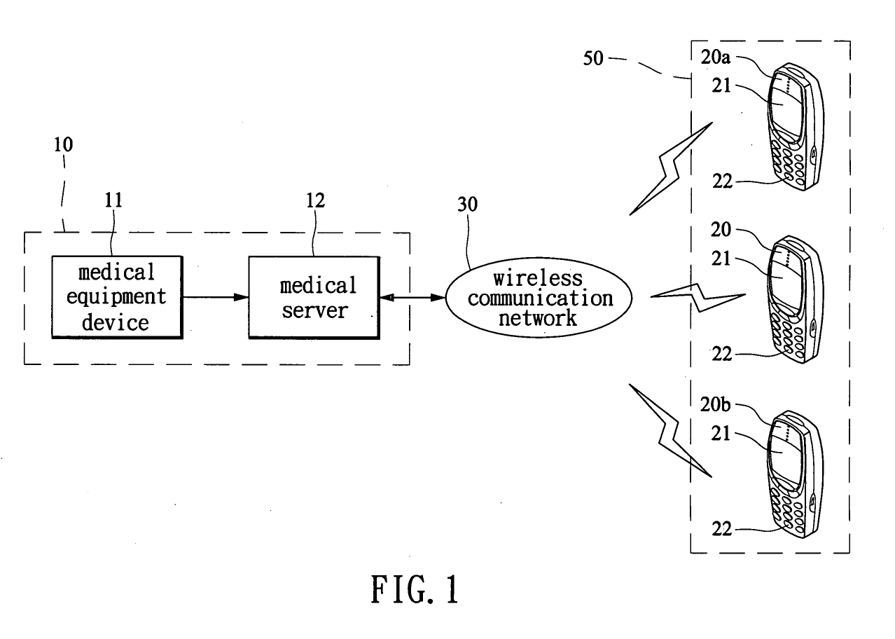

Reference is made to FIG. 1, which is a schematic diagram of the first embodiment in accord with the present invention. The system of the present invention includes a hospital end 10 and a remote end 50. The hospital end 10 has medical equipment device 11 and a medical server 12. The remote end 50 can employ multiple remote mobile communication apparatus. Here we take three remote mobile communication apparatuses 20, 20a, and 20b as example, but the amount of remote mobile communication apparatuses employed in the system should not be limited.

Each remote mobile communication apparatus 20, 20a, and 20b can be used to receive medical reports related to the patients of the hospital from the hospital end 10, in particularly speaking, from the medical server 12 at the hospital end 10. Every remote mobile communication apparatus 20, 20a, and 20b can provide a unique identification, such as a serial number, a phone number, an IP address, or a set of user account and password. When a remote mobile communication apparatus tries to connecting to the hospital end 10, the hospital end 10 can determine whether the it is authorized to access the medical server 12 via the unique identification. If the remote mobile communication apparatuses 20, 20a, and 20b are authorized to access the medical server 12, the doctor who uses the remote mobile communication apparatus 20 can remotely consulting with other doctors who hold the remote mobile communication apparatus 20a and the remote mobile communication apparatus 20b respectively via the medical server 12, though they might neither gather in the hospital nor even stay at the same location. The remote mobile communication apparatuses 20, 20a, and 20b can also send back the result of the consultation, i.e. modified medical reports generated during the consultation between the doctors, to the hospital end 10.

In the present invention, the remote mobile communication apparatus 20-20b are portable apparatuses, such as mobile phones or personal digital assistants (PDAs). The present invention takes a novel mobile phone for an example. The novel mobile phone has a larger screen 21. It can display an image message provided via the multimedia message service (MMS) or execute a Java program to download an image message. The present invention mainly uses the image displaying function of the mobile phone to show the medical reports on the screen 21 for remote consultation.

The medical equipment device 11 includes a medical image instrument and a medical report generator (not shown). The medical image instrument, for example, an angiographer, an ultrasound detector, an endoscopic camera, an intraoral camera or a tomography scanner, can be used to photograph an inner image of a human body to produce at least a medical image. On the other hand, the medical report generator, for example, a computer, is used to combine the medical images with medical text to generate a medical report.

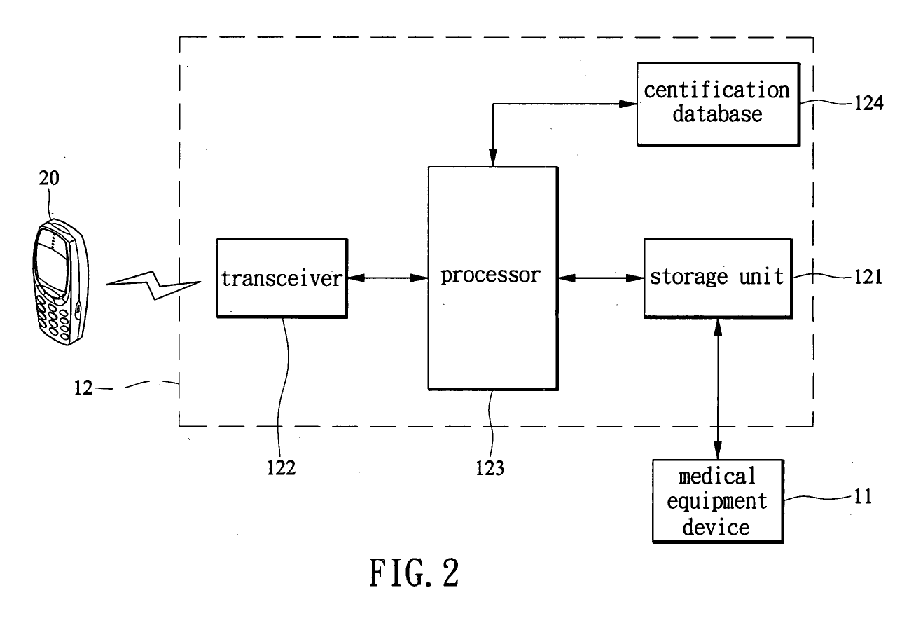

Reference is made to FIG. 1 together with FIG. 2, which is schematic diagram of an inner structure of the medical server 12 in accordance with the present invention. The medical server 12 is connected with the medical equipment device 11 for storing the medical reports generated by the medical equipment device 11, and storing the modified medical reports sent from the remote end 50. The medical server 12 primarily includes a storage unit 121, a transceiver 122, a processor 123, and a certification database 124.

The storage unit 121 can be used to store the medical reports from the medical equipment device 11 and from the remote end 50. The transceiver 122 can be used to send the original medical reports generated by the medical equipment device 11 to the remote mobile communication apparatus 20, receiving the modified medical reports sent from the remote mobile communication apparatus 20 via the wireless communication network 30, or send out the received modified medical reports again to another remote mobile communication apparatus 20a or 20b. Considering the importance of patients' privacy, the medical server 12 establishes the certification database 124 to record the identifications of those remote mobile communication apparatuses, doctors' information corresponding to the identifications, the authority of the doctors to access certain medical reports, and the sequences of the doctors in receiving or transmitting the medical reports they are authorized to access.

The processor 123 is connected with the storage unit 121, transceiver 122, and the certification database 124, for controlling the accessing, searching, storing or transmitting of the medical reports.

For example, a user, i.e. doctor, can use the remote mobile communication apparatus 20 to connect with the medical server 12 to access an original medical report (herein named as the first medical report) generated by the medical equipment device 11 for remote consultation. The processor 123 would certify the identification of the remote mobile communication apparatus 20 by comparing the phone number of the mobile phone with the recorded phone number in the certification database 124, verifying a set of account name and passwords the user input to the PDA with the data recorded in the certification database 124, or other similar ways. The processor 123 might also check if the doctor represented by remote mobile communication apparatus 20 is authorized to see the first medical report, such as determining the doctor in charge of the patient of the first medical report.

After the remote mobile communication apparatus 20 passes the certification process, the processor 123 would retrieve the first medical report from the storage unit 121, and transmit it through the transceiver 122 and the wireless communication network 30 to the remote mobile communication apparatus 20. Then the modified medical report (herein called the second medical report) can be sent back to the medical server 12 via the wireless communication network 30, too. The wireless communication network 30 can be a GSM system, GPRS system or 3G system. The remote mobile communication apparatus 20 can access the first medical report or send back the second medical report via employing the multimedia message service (MMS) or execute a Java program to download the first medical report or send back the second medical report.

Other doctors not stayed at the same physical place might also have to participate in consulting a patient's condition by observing the patient's medical report, such as before a surgery. Therefore, after the first medical report being downloaded or transmitted to the remote mobile communication apparatus 20, and then being modified as the second medical report and sent back to save in the medical server 12, other co-working doctors will receive the second medical report in a predetermined sequence.

Referring again to FIGS. 1 and 2, in this embodiment, the accessing sequence of the patent's medical report may be predetermined in the certification database 124. When the medical server 12 receives the second medical report sent from the remote mobile communication apparatus 20, the process 123 automatically retrieves the stored second medical report and sends it out immediately through the transceiver 122 and the wireless communication network 30 to the remote mobile communication apparatus 20a, according to the accessing sequence preset and recorded in the certification database 124.

The doctor holding the remote mobile communication apparatus 20a would receive the second medical report, which is previously modified from the original medical report, i.e. the first medical report, by the remote mobile communication apparatus 20. The second medical report shown on the remote mobile communication apparatus 20a may include the original inner image of the patient, and notes or descriptions added via the remote mobile communication apparatus 20. The second medical report can be modified again via the remote mobile communication apparatus 20a, such as adding additional notes, deleting previous notes, or making commends over the earlier descriptions, and so on. The remote mobile communication apparatus 20a then sends the modified second medical report (herein called third medical report) back to the medical server 12, certified the identification via the certification database 124, and saved in the storage unit 121.

The processor 123 immediately checks whether there are still other remote mobile communication apparatuses authorized to access the patient's medical report for consultation. If all of the authorized remote mobile communication apparatuses had received the patient's medical report and modifications had been made, the final version of the modified medical report of the patient would be stored in the storage unit 121, which can be transmitted again to all the authorized remote mobile communication apparatuses as a backup, or printed out for the surgery room, and so on. Otherwise, the third medical report can be transmitted to the remote mobile communication apparatus 20b, modified, and sent back just like remote mobile communication apparatus 20 or 20a does.

Each of the remote mobile communication apparatus 20, 20a, and 20b can include a screen 21 for displaying the medical report and an input unit 22 for browsing the medical report or modifying the medical image or text of the received medical report to form the modified medical report. The input unit 22 can include a magnifying, minifying, page-up or page-down key. Hence, a user can use the magnifying, minifying, page-up or page-down key to browse the medical images. Additionally, the user can also use text input keys or handwriting input device to input, increase or modify the medical text to form the modified medical report.

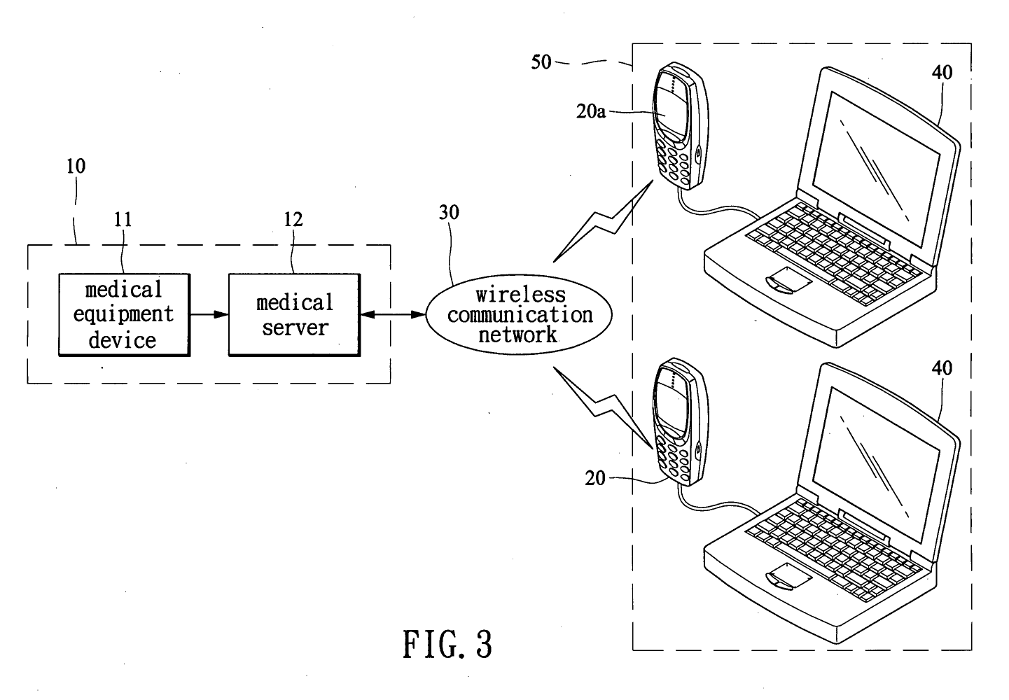

In the first embodiment of the present invention, although a doctor can use the remote mobile communication apparatus 20, such as a mobile phone or PDA, to compose the second medical report, the screen 21 is usually smaller than the size of the medical image and the functions of the input unit 22 may be limited, which may cause inconvenient for the user. Hence, the present invention connects the remote mobile communication apparatus 20 with a remote medical apparatus 40, as shown in FIG. 3, which is a schematic diagram of the second embodiment in accord with the present invention. The remote medical apparatus 40 can connect with the remote mobile communication apparatus 20 in a wireless or wired manner. Hence, a user can use it to access the medical report in the remote mobile communication apparatus 20, for example, the first medical report, generating the modified medical report and send the modified medical report back to the remote mobile communication apparatus 20, conveniently.

The remote medical apparatus 40 can be a computer, which mainly includes a medical report displaying device and a medical report generating device (not shown). The medical report displaying device can be used to browse the received and the modified medical report. Hence, it also includes the magnifying, minifying, page-up or page-down key. On the other hand, the medical report generating device can be used to increase, modify or vary the medical image or medical text of the received medical report to form, the modified medical report. The receiving or transmitting of the medical reports is still done by the remote mobile communication apparatus 20; the way of passing the modified medical report from one remote mobile communication apparatus to another through the medical server 12 can be referred to the first embodiment.

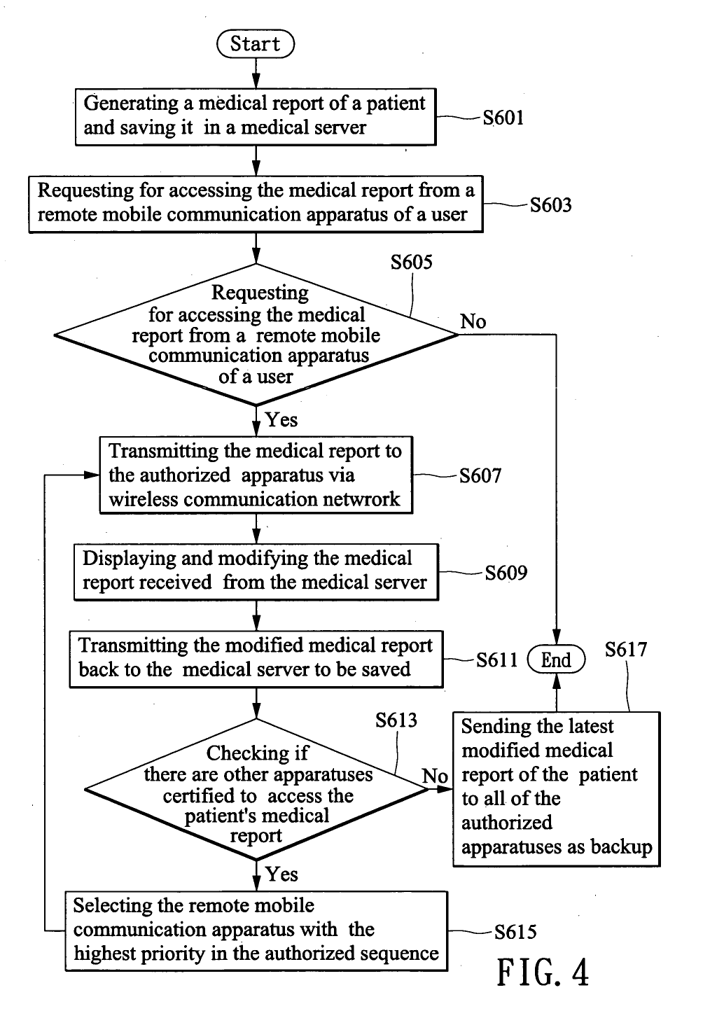

Referred to FIG. 4, which shows a flowchart of an embodiment of the method for remote consultation in accordance with the present invention. First of all, a medical equipment device 11 takes a patient's body inner image and generates an original medical report based on the inner image, and saves the report in a medical server 12 (S601). Once a user requests for accessing the stored original medical report via a remote mobile communication apparatus 20 (S603), the medical server 12 would certify whether the apparatus 20 is authorized to access the patient's medical report (S605). If the apparatus 20 is determined to be authorized to access the report, by comparing the identification of the apparatus 20 with a predetermined authorized accessing sequence, the original medical report would be transmitted to the remote mobile communication apparatus 20 via wireless communication network (S607); otherwise the request would be rejected.

The user can display the patient's medical report that received from the medical server 12 on the screen 21 of the remote mobile communication apparatus 20, and modify the medical report by using the input unit 22 of the apparatus 20 to generate a modified medical report (S609). Then the modified medical report is sent back from the apparatus 20 to the medical server 12 and saved (S611).

When the medical server 12 receives the modified medical report from the certified apparatus 20, the processor 123 of the server 12 checks whether there are other remote mobile communication apparatus also authorized to access the patient's report (S613), which means there might be other users, i.e. doctors, should know the comment made by the remote mobile communication apparatus 20 of the first doctor. When the medical server 12 determines there are other remote mobile communication apparatuses also authorized but not yet received the modified medical report, the medical server 12 selects the remote mobile communication apparatus with the highest priority in the authorized sequence (S615), and transmits the modified medical report that stored in the medical server 12 to the selected and certificated remote mobile communication apparatus (S607), for example, the remote mobile communication apparatus 20a, and repeats the following process as described above. Until the medical server 12 determines there is no other authorized remote mobile communication apparatus that is allowed to access the modified medical report of the patient, the medical server 12 can sent the latest modified medical report of the patient to all of the authorized apparatuses as backup (S617), and finish the remote consultation process.

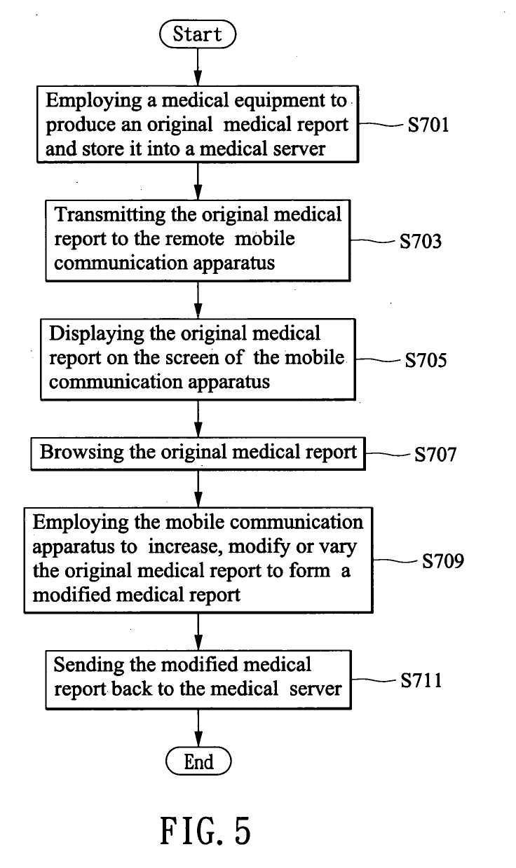

Reference is made to FIG. 5, which is a flowchart of the first embodiment that explain the detail of the medical report transmitting and receiving between the medical server and the remote mobile communication apparatus. In the present invention, the first embodiment includes following steps: first, employing the medical equipment device 11 to produce the original medical report and store it into the medical server 12 (S701); employing the medical server 12 to transmit the original medical report to the remote mobile communication apparatus 20 via the wireless communication network 30 (S703); displaying the original medical report on the screen 21 of the mobile communication apparatus 20 (S705); browsing the original medical report via the mobile communication apparatus 20 for remote consultation (S707); after consultation, employing the input unit 22 of the mobile communication apparatus 20 to increase, modify or vary the medical image or medical text of the original medical report to form the modified medical report (S709); and employing the mobile communication apparatus 20 to send the modified medical report back to the medical server 12 via the wireless communication network 30 (S711).

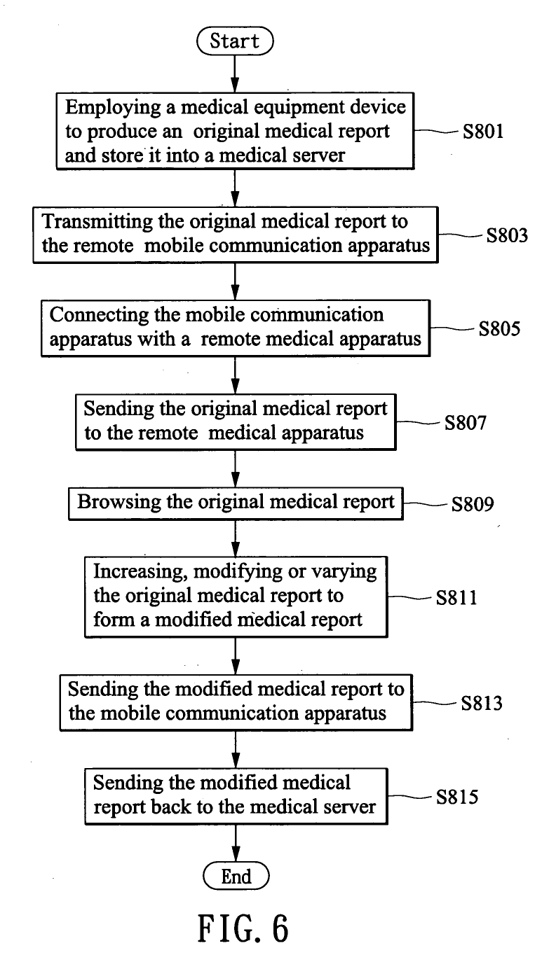

Reference is made to FIG. 6, which is a flowchart of the second embodiment that explain the detail of the medical report transmitted and received between the medical server and the remote mobile communication apparatus. In the present invention, the second embodiment includes following steps: first, employing the medical equipment device 11 to produce the original medical report and store it into the medical server 12 (S801); employing the medical server 12 to transmit the original medical report to the remote mobile communication apparatus 20 via the wireless communication network 30 (S803); connecting the mobile communication apparatus 20 with the remote medical apparatus 40 (S805); sending the original medical report to the remote medical apparatus 40 via the mobile communication apparatus 20 (S807); browsing the original medical report via the remote medical apparatus 40 for remote consultation (S809); after consultation, employing the remote medical apparatus 40 to increase, modify or vary the medical image or medical text of the original medical report to form the modified medical report (S811); sending the modified medical report to the mobile communication apparatus 20 (S813); and employing the mobile communication apparatus 20 to send the modified medical report back to the medical server 12 via the wireless communication network 30 (S815).

Although the present invention has been described with reference to the preferred embodiment thereof, it will be understood that the invention is not limited to the details thereof. Various substitutions and modifications have been suggested in the foregoing description, and other will occur to those of ordinary skill in the art. Therefore, all such substitutions and modifications are embraced within the scope of the invention as defined in the appended claims.

What is claimed is:

1. A system for remote consultation, comprising:
a medical server configured for storing multiple medical reports;
a medical equipment device operatively coupled with the medical server, used to capture an internal image of a patient and produce an original medical report, the original medical report containing the internal image of the patient and transmitted to the medical server; and
multiple remote mobile communication apparatuses respectively connected with the medical server via a wireless communication network, sequentially receiving one of the medical reports stored in the medical server, displaying the received medical report, and transmitting a modified medical report modified from the received medical report back to the medical server;
wherein each remote mobile communication apparatus is configured with an identification, and the medical reports that the remote mobile communication apparatuses received include the original medical report and the modified medical report.

2. The system as claimed in the claim 1, wherein the medical server comprises:
a storage unit used to store the medical reports;
a transceiver used to transmit the medical reports to the remote mobile communication apparatuses via the wireless communication network or receive the modified medical report from the remote mobile communication apparatuses;
a processor connected with the storage unit and the transceiver for accessing, searching, storing, or transmitting the medical reports; and
a certification database connected with the processor, recording the identifications of the remote mobile communication apparatuses;
wherein the processor accesses the certification database to certify the accessing authority and the accessing sequences of the remote mobile communication apparatuses via the identifications in accessing the medical reports stored in the storage unit.

3. The system as claimed in the claim 1, wherein the remote mobile communication apparatus comprises:
a screen for displaying the received medical report; and
an input unit for browsing the received medical report so as to increase, modify or vary a medical image or a medical text of the received medical report to form the modified medical report.

4. The system as claimed in the claim 1, further comprising:
a remote medical apparatus connected with one of the mobile communication apparatuses in a wired or a wireless manner, used to capture the medical report received by the remote mobile communication apparatus, and produce the modified medical report based on the received medical report.

5. The system as claimed in the claim 4, wherein the remote medical apparatus comprises:
a medical report displaying device used for a user to browse the medical reports; and
a medical report generating device used to increase, modify or vary a medical image or a medical text of the received medical report to form the modified medical report.

6. The system as claimed in the claim 1, wherein the medical equipment device has a medical image instrument used to photograph the internal image of a human body to produce at least a medical image.

7. The system as claimed in the claim 6, wherein the medical image instrument is an angiographer, an ultrasound detector, an endoscopic camera, an intraoral camera or a tomography scanner.

8. The system as claimed in the claim 1, wherein the medical equipment device has a medical report generator used to combine medical images with a medical text to generate the original medical report.

9. The system as claimed in the claim 8, wherein the medical report generator is a computer.

10. The system as claimed in the claim 1, wherein the original medical report comprises at least a medical image and a medical text.

11. The system as claimed in the claim 1, wherein the remote mobile communication apparatus is a portable apparatus.

12. The system as claimed in the claim 1, wherein the remote mobile communication apparatus uses a multimedia message service (MMS) to access the medical report or send the modified medical report back.

13. The system as claimed in the claim 1, wherein the remote mobile communication apparatus executes a Java program to download the medical report from the medical server or send the modified medical report back.

14. The system as claimed in the claim 3, wherein the input unit has a magnifying key, a minifying key, a page-up key or a page-down key.

15. The system as claimed in the claim 3, wherein the input unit is a text input key or handwriting input device.

16. The system as claimed in the claim 5, wherein the medical report displaying device has a magnifying key, a minifying key, a page-up key or a page-down key.

17. The system as claimed in the claim 5, wherein the remote medical apparatus is a computer.

18. A method for remote consultation, comprising:
a) providing a medical equipment device operatively coupled to a medical server that is used to stores multiple medical reports;
b) actuating the medical equipment device to capture an internal image of a patient;
c) generating an original medical report to include the internal image of the patient and storing the original medical report in the medical server;
d) employing the medical server to certify the accessing authority of a remote mobile communication apparatus via an identification of the remote mobile communication apparatus;
e) employing the medical server to transmit one of the medical reports to the remote mobile communication apparatus authorized to access the medical report via a wireless communication network;
f) displaying the medical report transmitted from the medical server on the authorized remote mobile communication apparatus, whereby a modified medical report is generated;
g) sending the modified medical report back to the medical server and storing the modified medical report;
h) the medical server automatically transmitting one of the medical reports to another remote mobile communication apparatus authorized to access the patient's medical report, wherein the medical report transmitted from the medical server is the modified medical report sent from the prior remote mobile communication apparatus;
i) employing step (f) and the following steps until the medical server determines that there is no more authorized remote mobile communication apparatuses.

19. The method as claimed in the claim 18, wherein after the step f) further comprises:
transmitting the transmitted medical report from the remote mobile communication apparatus to a remote medical apparatus; and
actuating the remote medical apparatus to display the transmitted medical report, whereby a modified medical report is produced.

20. The method as claimed in the claim 19, wherein the step of actuating the remote medical apparatus to display the transmitted medical report comprises:
consulting by using the original medical report and producing the modified medical report by using the remote medical apparatus.

21. The method as claimed in the claim 18, wherein the step c) further comprises:
employing the medical equipment device to produce the original medical report; and
storing the original medical report into the medical server.

22. The method as claimed in the claim 18, wherein the transmitted medical report of the step e) is the original medical report of the patient.

23. The method as claimed in the claim 18, wherein the step e) uses a multimedia message service (MMS) to transmit the original medical report.

24. The method as claimed in the claim 18, wherein the step e) executes a Java program to download the original medical report from the medical server.

25. The method as claimed in the claim 18, wherein the step f) displays the medical report on a screen of the remote mobile communication apparatus.

26. The method as claimed in the claim 18, wherein the step f) is performed by using an input unit of the remote mobile communication apparatus.

27. The method as claimed in the claim 26, wherein the step of displaying the medical report is performed by using a magnifying key, a minifying key, a page-up key or a page-down key of the input unit of the remote mobile communication apparatus.

28. The method as claimed in the claim 18, wherein the step f) is performed by using an input unit of the remote mobile communication apparatus to increase, modify or vary a medical image or a medical text of the medical report to form the modified medical report.
